(19) United States
(12) Patent Application Publication
(10) Pub. No.: US 2010/0184874 A1
(43) Pub. Date: Jul. 22, 2010

(54) FISCHER-TROPSCH PROCESS

(76) Inventors: Arend HOEK, Amsterdam (NL); Thomas Joris REMANS, Amsterdam (NL)

Correspondence Address: SHELL OIL COMPANY, P O BOX 2463, HOUSTON, TX, 772522463, US

(21) Appl. No.: 12/638,625

(22) Filed: Dec. 15, 2009

(30) Foreign Application Priority Data
Dec. 16, 2008 (EP) 08171752.2

(51) Int. Cl. C07C 27/06

(52) U.S. Cl. 518/712

(57) ABSTRACT

The present invention pertains to a Process for carrying out a high-speed stop in a Fischer-Tropsch process which comprises providing a feed comprising CO and H₂ to the inlet section of a fixed bed reactor comprising a Fischer-Tropsch catalyst, the reactor being at reaction temperature and pressure, and withdrawing an effluent from the outlet section of the reactor, wherein the high-speed stop is effected by blocking provision of the CO and H₂ to the reactor, and withdrawing gaseous reactor content from the reactor, the gaseous reactor content being withdrawn at least in part from the inlet section of the reactor.

This application claims the benefit of European Application No. 08171752.2 filed Dec. 16, 2008 which is incorporated herein by reference.

### BACKGROUND OF THE INVENTION

The present invention relates to a Fischer-Tropsch process, in particular to a process for carrying out a high-speed stop in a Fischer-Tropsch process carried out in a fixed bed reactor.

The Fischer-Tropsch process can be used for the conversion of hydrocarbonaceous feed stocks into normally liquid and/or solid hydrocarbons (0° C., 1 bar). The feed stock (e.g. natural gas, associated gas, coal-bed methane, residual oil fractions, biomass and/or coal) is converted in a first step into a mixture of hydrogen and carbon monoxide. This mixture is often referred to as synthesis gas or syngas. The synthesis gas is fed into a reactor where it is converted over a suitable catalyst at elevated temperature and pressure into paraffinic compounds ranging from methane to high molecular weight molecules comprising up to 200 carbon atoms, or, under particular circumstances, even more.

Numerous types of reactor systems have been developed for carrying out the Fischer-Tropsch reaction. For example, Fischer-Tropsch reactor systems include fixed bed reactors, especially multi-tubular fixed bed reactors, fluidised bed reactors, such as entrained fluidised bed reactors and fixed fluidised bed reactors, and slurry bed reactors such as three-phase slurry bubble columns and ebullated bed reactors.

The Fischer-Tropsch reaction is very exothermic and temperature sensitive. In consequence, careful temperature control is required to maintain optimum operation conditions and desired hydrocarbon product selectivity.

The fact that the reaction is very exothermic also has the consequence that when temperature control is not adequate, the reactor temperature can increase very quickly, which carries the risk of a reactor runaway, which may result in local deactivation of the catalyst.

The desired use of high-activity catalysts in Fischer-Tropsch fixed-bed reactors makes the situation even more challenging, because the susceptibility of a reactor to reactor runaway increases with increased catalyst activity. A reactor runaway is a most undesirable phenomenon, as it may result in catalyst deactivation which necessitates untimely replacement of the catalyst, causing reactor downtime and additional catalyst cost.

Therefore, there is need for a process for carrying out a high-speed stop in a Fischer-Tropsch reactor. A high-speed stop may, for example, be required when the temperature in the Fischer-Tropsch reactor increases to an unacceptable value either locally or over the entire reactor, when there is an interruption in the gas flow, or in the case of other unforeseen circumstances.

### SUMMARY OF THE INVENTION

Accordingly, the present invention pertains to a process for carrying out a high-speed stop in a Fischer-Tropsch process which comprises providing a feed comprising CO and H₂ to the inlet section of a fixed bed reactor comprising a Fischer-Tropsch catalyst, the reactor being at reaction temperature and pressure, and withdrawing an effluent from the outlet section of the reactor, wherein the high-speed stop is effected by blocking provision of CO and H₂ to the reactor, and withdrawing gaseous reactor content from the reactor, the gaseous reactor content being withdrawn at least in part from the inlet section of the reactor.

### DETAILED DESCRIPTION OF THE INVENTION

Depending on the process configuration, the performance of a high-speed stop in a Fischer-Tropsch reactor is often accompanied by a rise in temperature, culminating in a process-side temperature peak. This is caused by a decrease in gas hourly space velocity which leads to an increased conversion, accompanied by increased heat formation, and simultaneously to a decrease in heat removal capacity.

It was found that a high-speed stop according to the invention, where gaseous reactor content is withdrawn at least in part from the inlet section of the reactor, results in an increase in peak temperature which is substantially lower than the increase in peak temperature which is obtained when reactor content is withdrawn exclusively from the outlet section of the reactor. It was also found that the procedure according to the invention does not result in substantial catalyst deactivation.

The Fischer-Tropsch reactor comprises a catalyst section located between the inlet section of the reactor and the outlet section of the reactor. The inlet section of the reactor is provided with an inlet for the gaseous reactants, viz. hydrogen and CO, and optionally for inert gas to be added during the reaction or during the high-speed stop. As will be evident to the skilled person, the various components can be added to the reactor though the same or different inlets, depending on reactor configuration. The outlet section of the reactor is provided with an outlet for liquid product and an outlet for gaseous reactor content. Depending on reactor configuration, these outlets may be combined, or provided separately. The outlet section of the reactor may have more than one outlet for liquid product and/or more than one outlet for gaseous reactor content.

In the context of the present specification the wording bottom of the reactor refers to the part of the reactor below the part of the reactor where the catalyst is located. The wording top of the reactor refers to the part of the reactor above the part of the reactor where the catalyst is located. In the Fischer-Tropsch process according to the invention, the inlet section is generally provided at the top of the reactor. The outlet section is generally provided at the bottom of the reactor. It is, however, also possible to have the inlet section at the bottom of the reactor and the outlet section at the top.

The invention will be described in more detail below.

During the high-speed stop gaseous reactor content is withdrawn from the reactor. This reactor content encompasses gaseous reactants, gaseous products, and any inert gases added to the reactor during the reaction or during the high-speed stop. Depending on the reactor configuration liquid reaction products present in the unit may or may not be withdrawn from the reactor during the high-speed safety stop. As will be explained in more detail below, gaseous reactor content is withdrawn at least in part from the inlet section of the reactor. If liquid reaction products are withdrawn during the high-speed stop, this will generally be done from the outlet section. This is in particular the case when the inlet section is at the top of the reactor and the outlet section is at the bottom of the reactor.

In one embodiment of the present invention, gaseous reactor content is withdrawn only from the inlet section of the reactor, and not from the outlet section. In this embodiment the gas flow in the reactor during the high-speed stop will be reversed as compared to the gas flow during operation, namely from the outlet section to the inlet section as compared to from the inlet section to the outlet section. This has the advantage that the unreacted components present in the inlet section will not come into substantial contact with the catalyst. Gaseous reactor content present in the outlet section will pass through the catalyst section, but this material is less reactive than the content of the inlet section, due to a lower H₂/CO ratio, and possibly a higher content of inert gas. In conclusion, in this embodiment the amount of reactive component that passes through the catalyst section is reduced, which results in a reduction of the exotherm formed.

In this embodiment, the design of the reactor should be such that the reverse of the gas flow through the catalyst section can be accommodated. In particular, it may be necessary to secure the catalyst against lifting during the high-speed stop.

In this embodiment, liquid product present in the reactor may be retained in the reactor or withdrawn from the reactor, generally through the outlet section. It may be preferred to retain liquid product in the reactor.

In another embodiment of the present invention, during the high-speed stop gaseous reactor content is withdrawn from both the inlet section of the reactor and the outlet section of the reactor. In this case, the percentage of gaseous reactor content that is withdrawn from the inlet section of the reactor, calculated on the total of withdrawn gaseous reactor content may vary in wide ranges, for example, between 1 and 99 vol. %, more specifically between 10 and 90 vol. %. A value above 30% may be of particular interest.

Depending on the percentage of gaseous reactor content withdrawn from the inlet section, the gas flow in the catalyst section during the high-speed stop may be in the same direction as the gas flow during operation, or in the opposite direction. If the direction of the gas flow is reversed as compared to commercial operation, the design of the reactor should be such that this gas flow reverse can be accommodated.

The withdrawal of gaseous reactor content during the high-speed stop results in a reduction of the pressure in the reactor. The final pressure that is obtained is, generally, below 15 bar, more specifically in the range of 1-10 bar, for example in the range of 2-8 bar.

The reactor is generally operated before the high-speed temperature stop at an operating pressure which generally ranges from 5 to 150 bar, preferably from 20 to 80 bar, more in particular from 30 to 70 bar.

In the process of the invention, the provision of CO and H₂ to the reactor is stopped. In one embodiment an inert gas is added during the high-speed stop. The addition of inert gas may serve to help control the formation of an exotherm. Within the present specification, the term inert gas refers to a gas which is inert under Fischer-Tropsch reaction conditions. Examples of suitable inert gases include nitrogen and low-sulphur natural gas, for example desulphurised natural gas.

If used, inert gas may be added, for example in an amount of at least 5 Nl/l/h, more in particular at least 10 Nl/l/h, still more in particular at least 20 Nl/l/h. The amount of inert gas, if added, may for example be at most 80 Nl/l/h, more in particular at least 70 Nl/l/h, still more in particular at least 60 Nl/l/h.

The addition of inert gas is less preferred when the gas flow in the reactor is from the outlet section to the inlet section. This is because, in this case, the inert gas may be withdrawn from the unit before coming into contact with the catalyst. The addition of inert gas may be attractive in an embodiment where the gas flow in the reactor is from the inlet section to the outlet section.

If so desired, the reactor may be cooled during or after the high-speed stop. It is preferred to cool the reactor during the high speed stop. The end temperature of the cooling step depends on the desired further action. In general, the reactor will be cooled to a temperature between ambient and 200° C. Where the reactor is cooled with a view to immediate restarting of the reactor, it will generally be cooled to a temperature in the range of 100-190° C., in particular to a value of 160-180° C.

The cooling speed will depend on the size of the reactor and further circumstances. For example, it may be in the range of 10-100° C. per hour.

During operation of the Fischer-Tropsch process, the feed comprising CO and H₂ is provided to the inlet section of the reactor. The effluent is withdrawn from the outlet section of the reactor.

Depending on the design of the reactor, the effluent from the reactor during operation of the Fischer-Tropsch process can be a single gaseous phase, a multi-phase effluent or two or more effluent streams with one or more being mainly gaseous and one or more being mainly liquid phase.

The process according to the invention is suitable for fixed bed reactors. In a preferred embodiment the reactor is a reactor tube, which has a ratio between length and diameter of at least 5, in particular at least 50. As an upper limit a ratio of at most 1000 may be mentioned.

In one embodiment, the reactor tube is a tube in a multitubular reactor, which comprises a plurality of reactor tubes at least partially surrounded by a heat transfer medium.

The tubes in a multitubular reactor generally have a diameter in the range of 0.5-20 cm, more in particular in the range of 1 to 15 cm. They generally have a length in the range of 3 to 30 m. The number of tubes in a multitubular reactor is not critical to the present invention and may vary in wide ranges, for example in the range of 4 to 50,000, more in particular in the range of 100 to 40,000.

Multitubular reactors and their use in Fischer-Tropsch processes are known in the art and require no further elucidation here.

In one embodiment, the catalyst is a particulate catalyst, that is, a catalyst in the form of particles. The shape of the catalyst may be regular or irregular. The dimensions are suitably 0.1-30 mm in all three directions, preferably 0.1-20 mm in all three directions, more in particular 0.1-6 mm. Suitable shapes are spheres and, in particular, extrudates. The extrudates suitably have a length between 0.5 and 30 mm, preferably between 1 and 6 mm. The extrudates may be cylindrical, polylobal, or have any other shape. Their effective diameter, that is, the diameter of a sphere with the same outer surface over inner volume ratio, is suitably in the range of 0.1 to 10 mm, more in particular in the range of 0.2-6 mm.

A fairly recent trend in the development of Fischer-Tropsch catalysts is the development of catalyst particles with a decreased diffusion limitation. It has been found that catalysts with a decreased diffusion limitation are highly active in Fischer-Tropsch processes. However, due to their high activity, and their higher activation energy, their use entails an increased risk of reactor runaway. Further, it has also been found that catalysts with a decreased diffusion limitation are particularly sensitive to how a high-speed stop is carried out. More in particular, it has been found that for a catalyst with a decreased diffusion limitation a high-speed stop performed by blocking the flow of CO and H₂ to the reactor and depressurising the reactor via the bottom may lead to the formation of a temperature peak of the order of 100° C., which is difficult to address in commercial operation. On the other hand, when for the same decreased diffusion limitation catalyst the high-speed stop according to the invention was carried out, this resulted in an increase in peak temperature of the order of 23° C. Therefore, the process according to the invention is of particular interest for reactors comprising a catalyst with decreased diffusion limitation, in particular with an effective diameter of at most 2 mm, more in particular of at most 1.6 mm, still more in particular of at most 1.5 mm. Catalysts with a decreased diffusion limitation are for example described in WO2003/013725, WO2008/087149, WO2003/103833, and WO2004/041430.

The Fischer-Tropsch reaction is preferably carried out at a temperature in the range from 125 to 400° C., more preferably 175 to 300° C., most preferably 200 to 260° C. The gaseous hourly space velocity may vary within wide ranges and is typically in the range from 500 to 10000 Nl/l/h, preferably in the range from 1500 to 4000 Nl/l/h. The hydrogen to CO ratio of the feed as it is fed to the catalyst bed generally is in the range of 0.5:1 to 2:1.

Products of the Fischer-Tropsch synthesis may range from methane to heavy hydrocarbons. Preferably, the production of methane is minimized and a substantial portion of the hydrocarbons produced have a carbon chain length of a least 5 carbon atoms. Preferably, the amount of C5+ hydrocarbons is at least 60% by weight of the total product, more preferably, at least 70% by weight, even more preferably, at least 80% by weight, most preferably at least 85% by weight. The CO conversion of the overall process is preferably at least 50%.

The products obtained via the process according to the invention can be processed through hydrocarbon conversion and separation processes known in the art to obtain specific hydrocarbon fractions. Suitable processes are for instance hydrocracking, hydroisomerisation, hydrogenation and catalytic dewaxing. Specific hydrocarbon fractions are for instance LPG, naphtha, detergent feedstock, solvents, drilling fluids, kerosene, gasoil, base oil and waxes.

Fisher-Tropsch catalysts are known in the art. They typically comprise a Group VIII metal component, preferably cobalt, iron and/or ruthenium, more preferably cobalt. Typically, the catalysts comprise a catalyst carrier. The catalyst carrier is preferably porous, such as a porous inorganic refractory oxide, more preferably alumina, silica, titania, zirconia or combinations thereof. References to the Periodic Table and groups thereof used herein refer to the previous IUPAC version of the Periodic Table of Elements such as that described in the 68th Edition of the Handbook of Chemistry and Physics (CPC Press).

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material.

The catalytically active metal may be present in the catalyst together with one or more metal promoters or co-catalysts. The promoters may be present as metals or as the metal oxide, depending upon the particular promoter concerned. Suitable promoters include oxides of metals from Groups IA, IB, IVB, VB, VIB and/or VIIB of the Periodic Table, oxides of the lanthanides and/or the actinides. Preferably, the catalyst comprises at least one of an element in Group IVB, VB and/or VIIB of the Periodic Table, in particular titanium, zirconium, manganese and/or vanadium. As an alternative or in addition to the metal oxide promoter, the catalyst may comprise a metal promoter selected from Groups VIIB and/or VIII of the Periodic Table. Preferred metal promoters include rhenium, platinum and palladium.

A most suitable catalyst comprises cobalt as the catalytically active metal and zirconium as a promoter. Another most suitable catalyst comprises cobalt as the catalytically active metal and manganese and/or vanadium as a promoter. The promoter, if present in the catalyst, is typically present in an amount of from 0.1 to 60 parts by weight per 100 parts by weight of carrier material. It will however be appreciated that the optimum amount of promoter may vary for the respective elements which act as promoter. If the catalyst comprises cobalt as the catalytically active metal and manganese and/or vanadium as promoter, the cobalt:(manganese+vanadium) atomic ratio is advantageously at least 12:1.

It will be understood that it is within the scope of the skilled person to determine and select the most appropriate conditions for a specific reactor configuration and reaction regime.

The present invention is illustrated by the following examples, without being limited thereto or thereby.

### COMPARATIVE EXAMPLE

A Fischer-Tropsch process was operated at a temperature of about 220° C. and a pressure of about 40 bar in a fixed-bed reactor containing a catalyst.

A high-speed stop was carried out by blocking the flow of CO and H₂ to the reactor, while maintaining a nitrogen feed at an LHSV of 50 Nl/l/h. The reactor was depressurised via the bottom to a pressure of 20 barg in 6 minutes, and then to a pressure of 6 barg in an additional 14 minutes. The reactor temperature was measured during the high-speed stop, and a peak temperature of +100° C. above the maximum reaction temperature prior to the high-speed stop was measured.

Upon restart of the reactor it was found that no catalyst deactivation has taken place.

### Example According to the Invention

A Fischer-Tropsch process was operated at a temperature of about 220° C. and a pressure of about 40 bar in a fixed-bed reactor containing a catalyst.

A high-speed stop was carried out by blocking the flow of CO and H₂ to the reactor, while maintaining a nitrogen feed at an LHSV of 50 Nl/l/h. The reactor was depressurised via the top and bottom, with 30 vol. % of gas relieved via the top, calculated on the total of withdrawn gaseous reactor content. The depressurisation was carried out to a pressure of 20 barg in 6 minutes, and then to a pressure of 6 barg in an additional 14 minutes. The reactor temperature was measured during the high-speed stop, and a peak temperature of 36° C. above the maximum reaction temperature prior to the high-speed stop was measured.

Upon restart of the reactor it was found that no catalyst deactivation has taken place.

As compared to the procedure in the Comparative Example above, it appeared that the procedure according to the invention resulted in a substantially lower peak temperature.

What is claimed is:

1. A process for carrying out a high-speed stop in a Fischer-Tropsch process which comprises providing a feed comprising CO and H₂ to the inlet section of a fixed bed reactor comprising a Fischer-Tropsch catalyst, the reactor being at reaction temperature and pressure, and withdrawing an effluent from the outlet section of the reactor, wherein the high-speed stop is effected by blocking provision of CO and H₂ to the reactor, and withdrawing gaseous reactor content from the reactor, the gaseous reactor content being withdrawn at least in part from the inlet section of the reactor.

2. A process according to claim 1, wherein the gaseous reactor content is removed to reduce the reactor pressure to a value below 15 bar.

3. A process according to claim 1, wherein in the high-speed stop gaseous reactor content is withdrawn from the inlet section of the reactor and not from the outlet section.

4. A process according to claim 1, wherein in the high-speed stop gaseous reactor content is withdrawn from both the inlet section of the reactor and from the outlet section of the reactor.

5. A process according to claim 4, wherein the percentage of gaseous reactor content that is withdrawn from the inlet section of the reactor, calculated on the total of withdrawn gaseous reactor content is between 10 and 90 vol. %.

6. A process according to claim 1, wherein the inlet section is provided at the top of the reactor and the outlet section is provided at the bottom of the reactor.

7. A process according to claim 1, wherein the reactor comprises a reactor tube with a ratio between length and diameter of at least 50:1.

8. A process according to claim 7, wherein the reactor tube is a tube in a multitubular reactor, which comprises a plurality of reactor tubes at least partially surrounded by a heat transfer medium.

9. A process according to claim 1, wherein the catalyst is a particulate catalyst with an effective diameter of at most 1.6 mm, in particular at most 1.5 mm.

10. A process for carrying out a high-speed stop in a Fischer-Tropsch process which comprises providing a feed comprising CO and H₂ to the inlet section of a fixed bed reactor comprising a Fischer-Tropsch catalyst, the reactor being at reaction temperature and pressure, and withdrawing an effluent from the outlet section of the reactor, wherein the high-speed stop is effected by blocking provision of CO and H₂ to the reactor, and withdrawing gaseous reactor content from the reactor, the gaseous reactor content being withdrawn at least in part from the inlet section of the reactor wherein the percentage of gaseous reactor content that is withdrawn from the inlet section of the reactor, calculated on the total of withdrawn gaseous reactor content is between 10 and 90 vol. %.

11. A process according to claim 10, wherein the gaseous reactor content is removed to reduce the reactor pressure to a value below 15 bar.

12. A process according to claim 10, wherein the inlet section is provided at the top of the reactor and the outlet section is provided at the bottom of the reactor.

13. A process according to claim 10, wherein the reactor comprises a reactor tube with a ratio between length and diameter of at least 50:1.

14. A process according to claim 13, wherein the reactor tube is a tube in a multitubular reactor, which comprises a plurality of reactor tubes at least partially surrounded by a heat transfer medium.

15. A process for carrying out a high-speed stop in a Fischer-Tropsch process which comprises providing a feed comprising CO and H₂ to the inlet section of a fixed bed reactor comprising a Fischer-Tropsch catalyst, the reactor being at reaction temperature and pressure, and withdrawing an effluent from the outlet section of the reactor, wherein the high-speed stop is effected by blocking provision of CO and H₂ to the reactor, and withdrawing gaseous reactor content from the reactor, the gaseous reactor content being withdrawn at least in part from the inlet section of the reactor wherein the reactor comprises a reactor tube with a ratio between length and diameter of at least 50:1.

16. A process according to claim 15, wherein the gaseous reactor content is removed to reduce the reactor pressure to a value below 15 bar.

17. A process according to claim 15, wherein in the high-speed stop gaseous reactor content is withdrawn from the inlet section of the reactor and not from the outlet section.

18. A process according to claim 15, wherein in the high-speed stop gaseous reactor content is withdrawn from both the inlet section of the reactor and from the outlet section of the reactor.

19. A process according to claim 15, wherein the percentage of gaseous reactor content that is withdrawn from the inlet section of the reactor, calculated on the total of withdrawn gaseous reactor content is between 10 and 90 vol. %.

20. A process according to claim 15, wherein the reactor tube is a tube in a multitubular reactor, which comprises a plurality of reactor tubes at least partially surrounded by a heat transfer medium.
